(19) United States
(12) Patent Application Publication
(10) Pub. No.: US 2025/0360117 A1
(43) Pub. Date: Nov. 27, 2025

(54) KCNT1 INHIBITORS COMPRISING AN ISOXAZOLE OR OXADIAZOLE CORE AND METHODS OF USE

(71) Applicants: PRAXIS PRECISION MEDICINES, INC., Boston, MA (US); YALE UNIVERSITY, New Haven, CT (US)

(72) Inventors: SARAH HULLS, Boston, MA (US); MICHAEL KRISTOPHER MATHIEU KAHLIG, Redwood City, IA (US); ZOE HUGHES, Boston, MA (US); NELSON OLIVIER, Boston, MA (US); IMRAN QURAISHI, New Haven, CT (US); LEONARD KACZMAREK, New Haven, CT (US)

(73) Assignees: PRAXIS PRECISION MEDICINES, INC., Boston, MA (US); Yale University, New Haven, CT (US)

(21) Appl. No.: 18/872,629

(22) PCT Filed: Jun. 8, 2023
(86) PCT No.: PCT/US2023/024794

§ 371 Date: Dec. 6, 2024

Related U.S. Application Data
(60) Provisional application No. 63/350,325, filed on Jun. 8, 2022.

(51) Int. Cl. A61K 31/4439; A61P 25/08

(52) U.S. Cl. CPC A61K 31/4439; A61P 25/08

(57) ABSTRACT

Disclosed herein are compounds having an isoxazole core or an oxadiazole core or a pharmaceutically acceptable salt thereof and compositions useful for preventing and/or treating a neurological disorder, a disorder associated with excessive neuronal excitability, or disorder associated with a gain-of-function mutation in a gene (e.g., KCNT1). Methods of treating a neurological disorder, a disorder associated with excessive neuronal excitability, or disorder associated with a gain-of-function mutation in a gene, such as KCNT1, are also provided herein.

### CROSS-REFERENCE TO RELATED APPLICATION

This application claims priority to and the benefit of U.S. Provisional Patent Application No. 63/350,325, filed Jun. 8, 2022, the contents of which are incorporated herein by reference in their entirety.

### FIELD OF THE DISCLOSURE

The present disclosure is generally directed to KCNT1 inhibitors comprising an isoxazole core or an oxadiazole core, as well as pharmaceutical compositions and methods of treatment involving the use of such compounds.

### BACKGROUND OF THE DISCLOSURE

Potassium sodium-activated channel subfamily T member 1 (“KCNT1”) is one of the genes in a family of genes responsible for providing the instructions to make potassium channels. KCNT1 encodes sodium-activated potassium channels known as Slack (Sequence like a calcium-activated K⁺ channel). These channels are found in neurons throughout the brain and can mediate a sodium-activated potassium current I_{KNa}. This delayed outward current can regulate neuronal excitability and the rate of adaptation in response to maintained stimulation. Abnormal Slack activity has been associated with development of early onset epilepsies and intellectual impairment. Accordingly, pharmaceutical compounds that selectively regulate sodium-activated potassium channels, e.g., abnormal KCNT1 or abnormal I_{KNa}, are useful in treating a neurological disease or disorder or a disease or condition related to excessive neuronal excitability and/or KCNT1 gain-of-function mutations.

KCNT1 inhibitors and their preparation are disclosed, for example, in WO 2021/195066, incorporated herein by reference in its entirety. WO 2021/195066 discloses, for example, compounds with an isoxazole core having the Formula A:

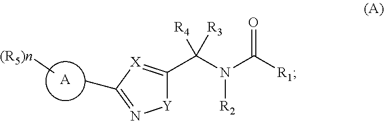

wherein X is CR₇ or N and Y is S; or
X is CR₇ and Y is O;
ring A is selected from the group consisting of phenyl, 6-membered heteroaryl, and 5-7 membered heteroaryl;
R₁ is selected from the group consisting of phenyl, 5-6 membered heteroaryl, —CH₂-phenyl, 5-8 membered carbocyclyl, and 5-10 membered heterocyclyl; wherein the phenyl 5-6 membered heteroaryl, —CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy; and C₃₋₈cycloakyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋₈cycloakylene or 3-7 membered heterocycloalkylene;
R₅ and R₆ are each independently selected from the group consisting of halogen, C₁₋₆alkyl, C₁₋₆alkylene-O—C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and —(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
   provided that when R₃ is hydrogen and ring A is a 6-membered heterocyclyl or 6-membered heteroaryl, R₁ is not thiophene; and
provided that when R₃ is hydrogen and ring A is a 6-membered heteroaryl or 5-membered heterocyclyl, R₁ is not phenyl; or
   a pharmaceutically acceptable salt thereof.

WO 2021/195066 also discloses several subgenera of Formula (A), including, for example, a compound of Formula I-IB having an isoxazole core:

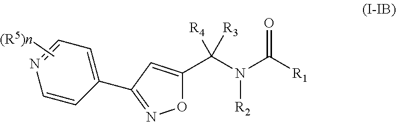

or a pharmaceutically acceptable salt thereof.

KCNT1 inhibitors and their preparation are disclosed, for example, in WO 2020/227101, incorporated herein by reference in its entirety. WO 2020/227101 discloses, for example, compounds with an oxadiazole core having the Formula (I):

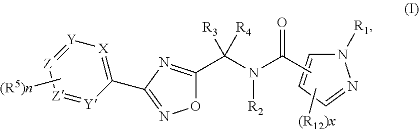

wherein X, Y, Z, Y′, and Z′ are each independently selected from CH and N, wherein the hydrogen of CH may be substituted with R₅, wherein at least 3 selected from X, Y, Z, Y′, and Z′ are CH;
R₁ is selected from the group consisting of C₁₋₆alkyl, C₃₋₁₀cycloalkyl, 3-10 membered heterocyclyl, 3-10 membered heteroaryl, and phenyl, wherein C₁₋₆alkyl, C₃₋₁₀cycloalkyl, 3-10 membered heterocyclyl, 3-10 membered heteroaryl, or phenyl is optionally substituted with one or more substituents each independently selected from the group consisting of halogen, C(O)N(R₉)₂, N(R₉)₂, C₃₋₇cycloalkyl, phenyl, 3-10 membered heteroaryl, and C₁₋₆alkoxy;
R₁₂ is selected from the group consisting of C₁₋₆alkyl, C₃₋₁₀cycloalkyl, 3-10 membered heterocyclyl, 3-10 membered heteroaryl, and phenyl, wherein the C₁₋₆alkyl, C₃₋₁₀cycloalkyl, 3-10 membered heterocyclyl, 3-10 membered heteroaryl, or phenyl is optionally substituted with one or more substituents each independently selected from the group consisting of halogen, —OH, —CN, C₁₋₆alkyl, C₁₋₆haloalkyl, and C₁₋₆alkoxy; or
two R₁₂ on adjacent carbons can be taken together with the two carbons where R₁₂ are attached to form a carbocyclic ring;
x is 0, 1, or 2;
R₂ is hydrogen or C₁₋₄alkyl;
R₃ is selected from the group consisting of hydrogen, C₁₋₆alkyl, C₃₋₁₀cycloalkyl, 3-10 membered heterocyclyl, 3-10 membered heteroaryl, and phenyl; and R₄ is selected from C₁₋₆alkyl and hydrogen; or R₃ and R₄ can be taken together with the carbon attached to R₃ and R₄ to form a C₃₋₇cycloalkylene or 3-7 membered heterocyclene; wherein the C₁₋₆alkyl, C₃₋₁₀cycloalkyl, 3-10 membered heterocyclyl, 3-10 membered heteroaryl, phenyl, C₃₋₇cycloalkylene, or 3-7 membered heterocyclene may be optionally substituted with one or more R₇;
each R₅ is independently selected from the group consisting of halogen, C₁₋₆alkyl, C₁₋₆haloalkyl, C₁₋₆alkylene-N(R₉)₂, C₁₋₆alkylene-O—C₃₋₁₀cycloalkyl, C₁₋₆alkoxy, C₁₋₆alkoxy substituted with C₃₋₁₀cycloalkyl optionally substituted with one or more halogens, C₁₋₆haloalkoxy, 3-10 membered heterocyclyl optionally substituted with one or more halogens or C₁₋₆alkoxy, 3-10 membered heteroaryl, C₁₋₆alkylene-OH, C₁₋₆alkylene-C₁₋₆alkoxy, OH, N(R₉)₂, —C(O)OR₈, C(O)N(R₉)₂, C₁₋₆alkylene-CN, —CN, —S(O)₂—C₁₋₆alkyl, C₁₋₆alkylene-S(O)₂—C₁₋₆alkyl, —S(O)₂—N(R₉)₂, —OC(O)C₁₋₆alkyl, —O—C₃₋₁₀cycloalkyl optionally substituted with one or more halogen or C₁₋₆alkyl, and C₃₋₁₀cycloalkyl optionally substituted with one or more substituents selected from halogen, C₁₋₆alkyl, and C₁₋₆alkoxy;
n is selected from the group consisting of 0, 1, 2, and 3;
R₇ is each independently selected from the group consisting of phenyl, C₁₋₆alkoxy, —OH, —N(R₉)₂, —NR₉—SO₂—C₁₋₆alkyl, —O—(C₁₋₆alkylene)-phenyl, C₃₋₁₀cycloalkyl, —C(O)OR₈, —C(O)N(R₉)₂, —NR₁₀C(O)—R₁₁, —CN, —S(O)₂—C₁₋₆alkyl, —S(O)₂—N(R₉)₂, 3-10 membered heterocyclyl, and 3-10 membered heteroaryl, wherein the phenyl, C₃₋₁₀cycloalkyl, 3-10 membered heterocyclyl, or 3-10 membered heteroaryl is optionally substituted with one or more substituents each independently selected from the group consisting of C₁₋₆alkyl, halogen, —OH, C₁₋₆alkoxy, and —N(R₉)₂;
R₈ is hydrogen or C₁₋₆alkyl;
each R₉ is independently selected from the group consisting of hydrogen, C₁₋₆alkyl, and —(C₁₋₆alkylene)-OH, or the two R₉ can be taken together with the nitrogen atom attached to the two R₉ to form a heterocycle optionally substituted with one or more substituents each independently selected from halogen and —OH;
each R₁₀ is independently hydrogen or C₁₋₆alkyl;
R₁₁ is selected from the group consisting of C₁₋₆alkyl, C₁₋₆alkoxy, and —O—(C₁₋₆alkylene)-phenyl; and
when R₃ and R₄ are both hydrogen, at least one selected from X, Y, Z, Y′, and Z′ is N, or a pharmaceutically acceptable salt thereof, and numerous species thereof.

### SUMMARY OF THE DISCLOSURE

Described herein are compounds with an isoxazole or an oxadiazole core and compositions useful for preventing and/or treating a disease, disorder, or condition, e.g., a neurological disorder, a disorder associated with excessive neuronal excitability, or disorder associated with a gain-of-function mutation in a gene, for example, KCNT1. In certain embodiments, the compounds and compositions may be useful for preventing and/or treating a disease, disorder, or condition of a fetus in utero.

In some aspects, provided is a compound of Formula (I-A) having an oxadiazole core:

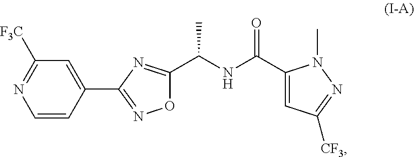 [SMILES: C[C@H](NC(=O)c1cc(C(F)(F)F)nn1C)c1nc(-c2ccnc(C(F)(F)F)c2)no1]

or a pharmaceutically acceptable salt thereof.

In other aspects, provided is a method of treating a neurological disorder, a disorder associated with excessive neuronal excitability, or a disorder associated with a gain-of-function mutation of a gene, by administering to a subject in need thereof an effective amount of a compound described herein having an isoxazole or an oxadiazole core, such as a compound of Formula (I-A), or a pharmaceutically acceptable salt thereof, or pharmaceutical compositions described herein comprising such compounds or a pharmaceutically acceptable salt thereof.

In some embodiments, the method provided involves treating a disorder associated with a gain-of-function mutation of KCNT1.

In some variations, the neurological disorder, the disorder associated with excessive neuronal excitability, or the disorder associated with a gain-of-function mutation of a gene (e.g., KCNT1) is epilepsy, an epilepsy syndrome, or an encephalopathy.

In some variations, the neurological disorder, the disorder associated with excessive neuronal excitability, or the disorder associated with a gain-of-function mutation of a gene (e.g., KCNT1) is a genetic or pediatric epilepsy or a genetic or pediatric epilepsy syndrome.

In some variations, the neurological disorder, the disorder associated with excessive neuronal excitability, or the disorder associated with a gain-of-function mutation of a gene (e.g., KCNT1) is a cardiac dysfunction.

In some variations, the neurological disorder, the disorder associated with excessive neuronal excitability, or the disorder associated with a gain-of-function mutation of a gene (e.g., KCNT1) is selected from the group consisting of epilepsy and other encephalopathies, malignant migrating focal seizures of infancy (MMFSI) or epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, or cerebellar ataxia.

In some variations, the neurological disorder, the disorder associated with excessive neuronal excitability, or the disorder associated with a gain-of-function mutation of a gene (e.g., KCNT1) is chosen from cardiac arrhythmia, Brugada syndrome, or myocardial infarction.

In some variations, the neurological disorder, the disorder associated with excessive neuronal excitability, or the disorder associated with a gain-of-function mutation of a gene (e.g., KCNT1) is selected from pain and related conditions (e.g., neuropathic pain, acute/chronic pain, migraine).

In some variations, the neurological disorder, the disorder associated with excessive neuronal excitability, or the disorder associated with a gain-of-function mutation of a gene (e.g., KCNT1) is a muscle disorder (e.g., myotonia, neuromyotonia, cramp muscle spasms, spasticity).

In some variations, the neurological disorder, the disorder associated with excessive neuronal excitability, or the disorder associated with a gain-of-function mutation of a gene (e.g., KCNT1) is selected from itch and pruritis, ataxia, or cerebellar ataxias.

In some variations, the neurological disorder, the disorder associated with excessive neuronal excitability, or the disorder associated with a gain-of-function mutation of a gene (e.g., KCNT1) is a psychiatric disorder (e.g., major depression, anxiety, bipolar disorder, schizophrenia).

In other variations, the neurological disorder, the disorder associated with excessive neuronal excitability, or the disorder associated with a gain-of-function mutation in a gene (e.g., KCNT1) is chosen from a learning disorder, Fragile X, neuronal plasticity, or an autism spectrum disorder.

In yet other variations, the neurological disorder, the disorder associated with excessive neuronal excitability, or the disorder associated with a gain-of-function mutation of a gene (e.g., KCNT1) is chosen from epileptic encephalopathy with SCN1A, SCN2A, and/or SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, sudden unexpected death in epilepsy (SUDEP), or KCNT1 epileptic encephalopathy.

In some variations, the subject is a human and the neurological disorder, the disorder associated with excessive neuronal excitability, or the disorder associated with a gain-of-function mutation of a gene is a R474H mutation in KCNT1, and in some variations, the R474H mutation in KCNT1 is a heterozygous mutation

In some variations, the subject is in utero and the compound of Formula (I-A) or a pharmaceutically acceptable salt thereof is administered to a pregnant mother of the subject, and in some variations, the method further comprises administering the compound of Formula (I-A) or a pharmaceutically acceptable salt thereof to the subject following birth.

Other objects and advantages will become apparent to those skilled in the art from consideration of the ensuing description.

### BRIEF DESCRIPTION OF THE FIGURES

FIG. 1A is a plot showing the latency to seizure in Kcnt1^{R455H/+} heterozygous mice administered a vehicle control and Formula (I-A) for 1 hour of pretreatment before administration of pentylenetetrazole (PTZ), as described in Example 1.

FIG. 1B is a plot showing the latency to seizure in Kcnt1^{R455H/+} heterozygous mice administered a vehicle control and Formula (I-A) for 2 hours of pretreatment before administration of PTZ, as described in Example 1.

FIG. 2 are graphs showing the individual interictal spike rate in Kcnt1^{R455H/+} heterozygous mice over a 6-day period including 3 days before and 3 days after administration of Formula (I-A), as described in Example 2.

FIG. 3A is a graph showing the daily averaged interictal spike rate in Kcnt1^{R455H/+} heterozygous mice from the 72-hour baseline and 1, 2, and 3 days after administration of Formula (I-A), as described in Example 2.

FIG. 3B is a graph showing the baseline-adjusted averaged interictal spike rate in Kcnt1^{R455H/+} heterozygous mice from the 72-hour baseline and 1, 2, and 3 days after administration of Formula (I-A), as described in Example 2.

### DESCRIPTION OF THE DISCLOSURE

Provided herein, in certain aspects, are compounds and compositions useful for preventing and/or treating a disease, disorder, or condition described herein, e.g., a neurological disorder, a disorder associated with excessive neuronal excitability, or a disorder associated with gain-of-function mutations in a gene (e.g., KCNT1). Exemplary diseases, disorders, or conditions include epilepsy and other encephalopathies (e.g., MMFSI or EIMFS, ADNFLE (now known as SHE), West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, Intellectual disability, Multifocal Epilepsy, Generalized tonic clonic seizures, Drug resistant epilepsy, Temporal lobe epilepsy, cerebellar ataxia, Asymmetric Tonic Seizures); cardiac dysfunctions (e.g., cardiac arrhythmia, Brugada syndrome, myocardial infarction); pain and related conditions (e.g., neuropathic pain, acute/chronic pain, migraine, etc.); muscle disorders (e.g., myotonia, neuromyotonia, cramp muscle spasms, spasticity); itch and pruritis; ataxia and cerebellar ataxias; and psychiatric disorders (e.g., major depression, anxiety, bipolar disorder, schizophrenia).

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

The terms “disease,” “disorder,” and “condition” are used interchangeably herein.

As used herein, the term “in some embodiments,” “in other embodiments,” or the like, refers to embodiments of all aspects of the disclosure, unless the context clearly indicates otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described, for example, in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The following terms are intended to have the meanings presented therewith below and are useful in understanding the description and intended scope of the present disclosure. When describing certain aspects of the disclosure, which may include compounds, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to. 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Hetero” when used to describe a compound or a group present on a compound means that one or more carbon atoms in the compound or group have been replaced by a nitrogen, oxygen, or sulfur heteroatom. Hetero may be applied to any of the alkyl groups described above such as alkyl, e.g., heteroalkyl; alkenyl, e.g., heteroalkenyl; alkynyl, e.g., heteroalkynyl; carbocyclyl, e.g., heterocyclyl; aryl, e.g., heteroaryl, and the like having from 1 to 5, and particularly from 1 to 3 heteroatoms.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₅ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spire ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Cyano” refers to —CN.

“Halo” or “halogen” refers to a fluorine atom (i.e., fluoro or —F), a chlorine atom (i.e., chloro or —Cl), a bromine atom (i.e., bromo or —Br), and an iodine atom (i.e., iodo or —I). In certain embodiments, the halo group is fluoro or chloro.

“Haloalkyl” refers to an alkyl group substituted with one or more halogen atoms.

“Nitro” refers to —NO₂.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. The general concept of pharmaceutically acceptable salts has been discussed in the art, including, for example, Berge et al., which describes pharmaceutically acceptable salts in detail in J Pharmaceutical Sciences (1977) 66:1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “modified-release polymer” refers to a polymer that is used in a formulation (e.g., tablets and capsules) to modify the release rate of the drug upon administration to a subject. For example, a modified-release polymer is used to dissolve a drug over time in order to be released slower and steadier into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, hydroxyl-propyl methylcellulose (HPMC)), hydrophobic matrix polymers (e.g., ethyl cellulose, ethocel), or polyacrylate polymers (e.g., Eudragit® RL100, Eudragit® RS100).

The term “diluent” as used herein refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), sugar alcohols (e.g., sorbitol, xylitol and mannitol)).

The term “glidant” as used herein refers to an excipient used to promote powder flow by reducing interparticle friction and cohesion. For example, glidants include fumed silica (e.g., colloidal silicon dioxide), talc, and magnesium carbonate.

The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

The term “coating” as used herein refers to an excipient to protect tablet ingredients from deterioration by moisture in the air and make large or unpleasant-tasting tablets easier to swallow.

The embodiments disclosed herein are not intended to be limited in any manner by the above exemplary listing of chemical groups and substituents. Those skilled in the art will recognize that several embodiments are possible within the scope and spirit of the present disclosure. The following description illustrates the disclosure and, of course, should not be construed in any way as limiting the scope of the inventions described herein.

### II. Compounds and Compositions

In one aspect, provided herein is a compound of Formula I:

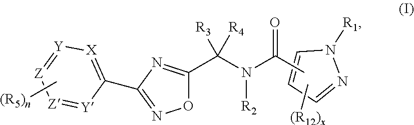

or a pharmaceutically acceptable salt thereof, for use in the methods described herein, wherein
X, Y, Z, Y′, and Z′ are each independently selected from CH and N, wherein the hydrogen of CH may be substituted with R₅, wherein at least 3 selected from X, Y, Z, Y′, and Z′ are CH;
R₁ is selected from the group consisting of C₁₋₆alkyl, C₃₋₁₀cycloalkyl, 3-10 membered heterocyclyl, 3-10 membered heteroaryl, and phenyl, wherein C₁₋₆alkyl, C₃₋₁₀cycloalkyl, 3-10 membered heterocyclyl, 3-10 membered heteroaryl, or phenyl is optionally substituted with one or more substituents each independently selected from the group consisting of halogen, C(O)N(R₉)₂, N(R₉)₂, C₃₋₇cycloalkyl, phenyl, 3-10 membered heteroaryl, and C₁₋₆alkoxy;
R₁₂ is selected from the group consisting of C₁₋₆alkyl, C₃₋₁₀cycloalkyl, 3-10 membered heterocyclyl, 3-10 membered heteroaryl, and phenyl, wherein the C₁₋₆alkyl, C₃₋₁₀cycloalkyl, 3-10 membered heterocyclyl, 3-10 membered heteroaryl, or phenyl is optionally substituted with one or more substituents each independently selected from the group consisting of halogen, —OH, —CN, C₁₋₆alkyl, C₁₋₆haloalkyl, and C₁₋₆alkoxy; or
two R₁₂ on adjacent carbons can be taken together with the two carbons where R₁₂ are attached to form a carbocyclic ring;
x is 0, 1 or 2;
R₂ is hydrogen or C₁₋₄alkyl;
R₃ is selected from the group consisting of hydrogen, C₁₋₆alkyl, C₃₋₁₀ cycloalkyl, 3-10 membered heterocyclyl, 3-10 membered heteroaryl, and phenyl; and R₄ is selected from C₁₋₆alkyl and hydrogen; or R₃ and R₄ can be taken together with the carbon attached to R₃ and R₄ to form a C₃₋₇cycloalkylene or 3-7 membered heterocyclene; wherein the C₁₋₆alkyl, C₃₋₁₀ cycloalkyl, 3-10 membered heterocyclyl, 3-10 membered heteroaryl, phenyl, C₃₋₇cycloalkylene, or 3-7 membered heterocyclene may be optionally substituted with one or more R₇;
each R₅ is independently selected from the group consisting of halogen, C₁₋₆alkyl, C₁₋₆haloalkyl, C₁₋₆alkylene-N(R₉)₂, C₁₋₆alkylene-O—C₃₋₁₀cycloalkyl, C₁₋₆alkoxy, C₁₋₆alkoxy substituted with C₃₋₁₀cycloalkyl optionally substituted with one or more halogens, C₁₋₆haloalkoxy, 3-10 membered heterocyclyl optionally substituted with one or more halogens or C₁₋₆alkoxy, 3-10 membered heteroaryl, C₁₋₆alkylene-OH, C₁₋₆alkylene-C₁₋₆alkoxy, OH, N(R₉)₂, —C(O)OR₈, C(O)N(R₉)₂, C₁₋₆alkylene-CN, —CN, —S(O)₂—C₁₋₆alkyl, C₁₋₆alkylene-S(O)₂—C₁₋₆alkyl, —S(O)₂—N(R₉)₂, —OC(O)C₁₋₆alkyl, —O—C₃₋₁₀cycloalkyl optionally substituted with one or more halogen or C₁₋₆alkyl, and C₃₋₁₀cycloalkyl optionally substituted with one or more substituents selected from halogen, C₁₋₆alkyl, and C₁₋₆alkoxy;
n is selected from the group consisting of 0, 1, 2, and 3;
R₇ is each independently selected from the group consisting of phenyl, C₁₋₆alkoxy, —OH, —N(R₉)₂, —NR₉—SO₂—C₁₋₆alkyl, —O—(C₁₋₆alkylene)-phenyl, C₃₋₁₀cycloalkyl, —C(O)OR₈, —C(O)N(R₉)₂, —NR₁₀C(O)—R₁₁, —CN, —S(O)₂—C₁₋₆alkyl, —S(O)₂—N(R₉)₂, 3-10 membered heterocyclyl, and 3-10 membered heteroaryl, wherein the phenyl, C₃₋₁₀cycloalkyl, 3-10 membered heterocyclyl, or 3-10 membered heteroaryl is optionally substituted with one or more substituents each independently selected from the group consisting of C₁₋₆alkyl, halogen, —OH, C₁₋₆alkoxy, and —N(R₉)₂;
R₈ is hydrogen or C₁₋₆alkyl;
each R₉ is independently selected from the group consisting of hydrogen, C₁₋₆alkyl, and —(C₁₋₆alkylene)-OH, or the two R₉ can be taken together with the nitrogen atom attached to the two R₉ to form a heterocycle optionally substituted with one or more substituents each independently selected from halogen and —OH;
each R₁₀ is independently hydrogen or C₁₋₆alkyl;
R₁₁ is selected from the group consisting of C₁₋₆alkyl, C₁₋₆alkoxy, and —O—(C₁₋₆alkylene)-phenyl; and
when R₃ and R₄ are both hydrogen, at least one selected from X, Y, Z, Y′, and Z′ is N.

In some embodiments, the compound is a compound of Formula I-I or Formula I-II:

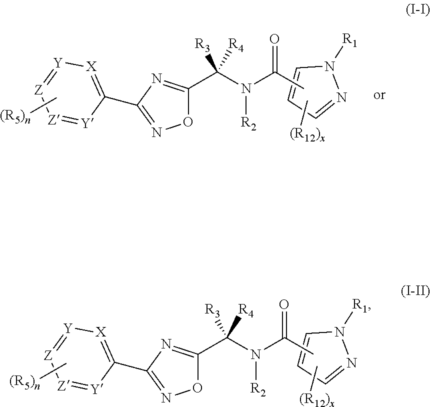

or a pharmaceutically acceptable salt thereof, wherein the variables are as defined above.

In some embodiments, one of X, Y, Z, Y′, and Z′ is N and the other four are CH.

In some embodiments, two of X, Y, Z, Y′, and Z′ are N and the other three are CH.

In some embodiments, the compound is a compound of Formula I-a:

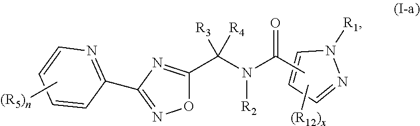

or a pharmaceutically acceptable salt thereof, wherein the variables are as defined above.

In some embodiments, the compound is a compound of Formula I-b:

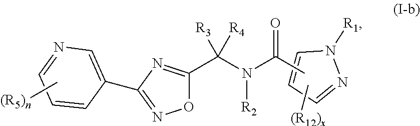

or a pharmaceutically acceptable salt thereof, wherein the variables are as defined above.

In some embodiments, the compound is a compound of Formula I-c:

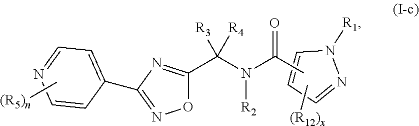

or a pharmaceutically acceptable salt thereof, wherein the variables are as defined above.

In some embodiments, the compound is a compound of Formula I-d:

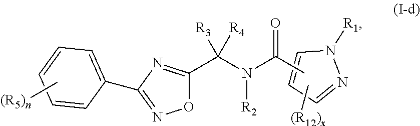

or a pharmaceutically acceptable salt thereof, wherein the variables are as defined above.

In some embodiments, the compound is a compound of Formula I-e:

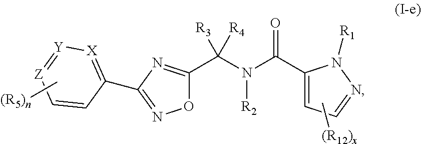

or a pharmaceutically acceptable salt thereof, wherein the variables are as defined above.

In some embodiments, the compound is a compound of Formula I-f:

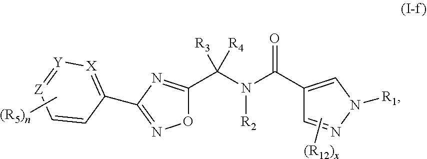

or a pharmaceutically acceptable salt thereof, wherein the variables are as defined above.

In some embodiments, the compound is a compound of Formula I-g:

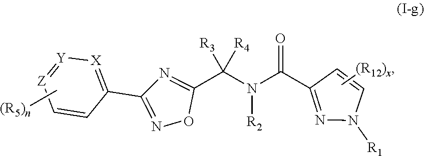

or a pharmaceutically acceptable salt thereof, wherein the variables are as defined above.

In some embodiments, the compound is a compound of Formula I-h:

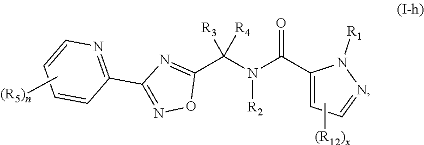

or a pharmaceutically acceptable salt thereof, wherein the variables are as defined above.

In some embodiments, the compound is a compound of Formula I-i:

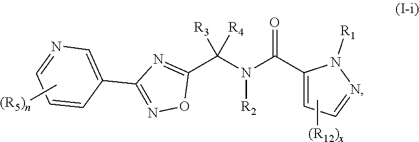

or a pharmaceutically acceptable salt thereof, wherein the variables are as defined above.

In some embodiments, the compound is a compound of Formula I-j:

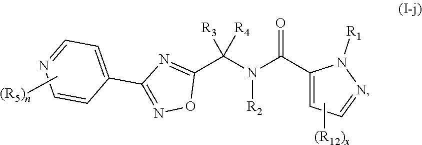

or a pharmaceutically acceptable salt thereof, wherein the variables are as defined above.

In some embodiments, the compound is a compound of Formula I-k:

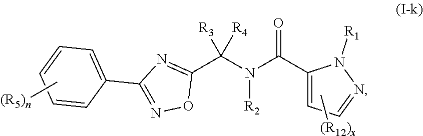

or a pharmaceutically acceptable salt thereof, wherein the variables are as defined above.

In some embodiments, the compound is a compound of Formula I-1:

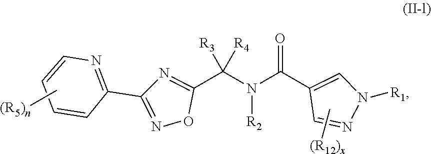

or a pharmaceutically acceptable salt thereof, wherein the variables are as defined above.

In some embodiments, the compound is a compound of Formula I-m:

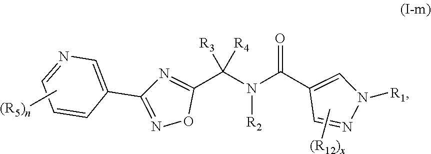

or a pharmaceutically acceptable salt thereof, wherein the variables are as defined above.

In some embodiments, the compound is a compound of Formula I-n:

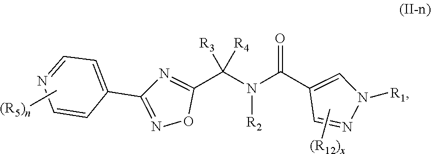

or a pharmaceutically acceptable salt thereof, wherein the variables are as defined above.

In some embodiments, the compound is a compound of Formula I-o:

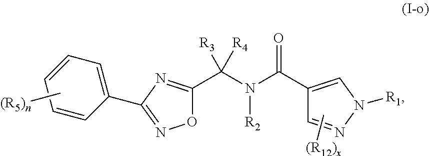

or a pharmaceutically acceptable salt thereof, wherein the variables are as defined above.

In some embodiments, the compound is a compound of Formula I-p:

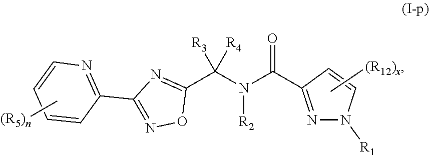

or a pharmaceutically acceptable salt thereof, wherein the variables are as defined above.

In some embodiments, the compound is a compound of Formula I-q:

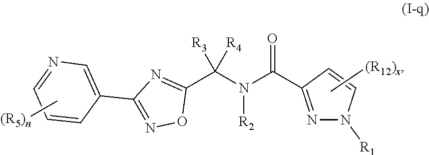

or a pharmaceutically acceptable salt thereof, wherein the variables are as defined above.

In some embodiments, the compound is a compound of Formula I-r:

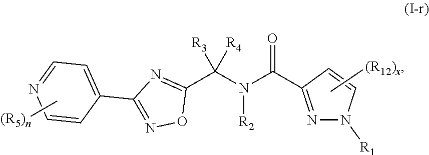

or a pharmaceutically acceptable salt thereof, wherein the variables are as defined above.

In some embodiments, the compound is a compound of Formula I-s:

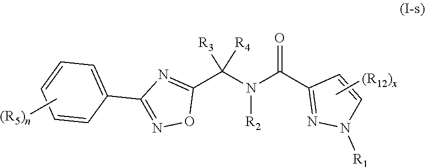

or a pharmaceutically acceptable salt thereof, wherein the variables are as defined above.

In one aspect, provided is a compound of Formula (I-A) having an oxadiazole core:

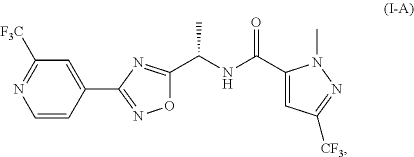 [SMILES: C[C@H](NC(=O)c1cc(C(F)(F)F)nn1C)c1nc(-c2ccnc(C(F)(F)F)c2)no1]

Or a stereoisomer thereof, or a pharmaceutically acceptable salt thereof, for use in the methods described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Embodiments disclosed herein additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, such as more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight, or more than 99.9% by weight, of the enantiomer. In certain embodiments, the weights are based upon total weight of all enantiomers or stereoisomers of the compound.

In certain aspects, provided are compositions comprising the compounds described herein. In some embodiments, an enantiomerically pure compound can be present in the compositions with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise at least about 95% by weight S-compound and at most about 5% by weight R-compound, by total weight of the compound. In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C. O may be in any isotopic form, including ¹⁶O and ¹⁸O, and F may be in any isotopic form, including ¹⁸F and ¹⁹F.

### III. Methods of Treatment

The compounds and compositions described above and herein can be used to treat a neurological disorder, a disorder associated with excessive neuronal excitability, or a disorder associated with a gain-of-function mutation in a gene (e.g., KCNT1). It is understood that any of the methods of treatment disclosed herein can be converted to the corresponding medical use or Swiss-type format. Thus, the present disclosure also provides any of the compounds or compositions described herein for use in treating a neurological disorder, a disorder associated with excessive neuronal excitability, or a disorder associated with a gain-of-function mutation in a gene (e.g., KCNT1). Also disclosed is any of the compounds or compositions as described herein, for the manufacture of a medicament for use in treating a neurological disorder, a disorder associated with excessive neuronal excitability, or a disorder associated with a gain-of-function mutation in a gene (e.g., KCNT1).

In some aspects, provided are methods of treating a neurological disorder, a disorder associated with excessive neuronal excitability, or a disorder associated with a gain-of-function mutation of a gene, by administering to a subject in need thereof an effective amount of any of the compounds described herein or a pharmaceutically acceptable salt thereof, or pharmaceutical compositions comprising such compounds or a pharmaceutically acceptable salt thereof.

Exemplary diseases, disorders, or conditions include epilepsy and other encephalopathies (e.g., MMFSI or EIMFS, ADNFLE, West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy, Lennox-Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, and leukoencephalopathy), SUDEP, cardiac dysfunctions (e.g., cardiac arrhythmia, Brugada syndrome, myocardial infarction), pulmonary vasculopathy/hemorrhage, pain and related conditions (e.g., neuropathic pain, acute/chronic pain, migraine, etc.), muscle disorders (e.g., myotonia, neuromyotonia, cramp muscle spasms, spasticity), itch and pruritis, movement disorders (e.g., ataxia and cerebellar ataxias), psychiatric disorders (e.g., major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

In some embodiments, the neurological disorder, the disorder associated with excessive neuronal excitability, or the disorder associated with a gain-of-function mutation in a gene (e.g., KCNT1) is selected from EIMFS, ADNFLE, or West syndrome. In some embodiments, the neurological disorder, the disorder associated with excessive neuronal excitability, or the disorder associated with a gain-of-function mutation in a gene (e.g., KCNT1) is selected from infantile spasms, epileptic encephalopathy, focal epilepsy, Ohtahara syndrome, developmental and epileptic encephalopathy, or Lennox-Gastaut syndrome. In some embodiments, the neurological disorder, the disorder associated with excessive neuronal excitability, or the disorder associated with a gain-of-function mutation in a gene (e.g., KCNT1) is seizure. In some embodiments, the neurological disorder, the disorder associated with excessive neuronal excitability, or the disorder associated with a gain-of-function mutation in a gene (e.g., KCNT1) is selected from cardiac arrhythmia, Brugada syndrome, or myocardial infarction.

In some embodiments, the neurological disorder, the disorder associated with excessive neuronal excitability, or the disorder associated with a gain-of-function mutation in a gene (e.g., KCNT1) is selected from a learning disorder, Fragile X, intellectual function, neuronal plasticity, a psychiatric disorder, or an autism spectrum disorder.

Accordingly, the compounds, pharmaceutically acceptable salts thereof, and compositions disclosed herein can be administered to a subject with a neurological disorder, a disorder associated with excessive neuronal excitability, or a disorder associated with a gain-of-function mutation in a gene such as KCNT1 (e.g., EIMFS, ADNFLE, West syndrome, infantile spasms, epileptic encephalopathy, focal epilepsy, Ohtahara syndrome, developmental and epileptic encephalopathy, Lennox-Gastaut syndrome, seizures, cardiac arrhythmia, Brugada syndrome, and myocardial infarction).

In certain embodiments, the compounds, pharmaceutically acceptable salts thereof, and compositions disclosed herein can be administered to a human subject with a mutation at amino acid residue R474. In certain embodiments, mutation at amino acid residue R474 is an R474H mutation. In certain embodiments, the human subject is heterozygous for the mutation at amino acid residue R474, such as the R474H mutation.

In certain embodiments, the compounds, pharmaceutically acceptable salts thereof, and compositions disclosed herein can be administered to a subject while the subject is in utero, e.g., by administering the compound, pharmaceutically acceptable salt thereof, or composition to a pregnant mother of a subject. In certain embodiments, the compound, pharmaceutically acceptable salt thereof, or composition is administered to a subject following birth, wherein the subject was treated the compound, pharmaceutically acceptable salt thereof, or composition in utero. In certain embodiments, the compound, pharmaceutically acceptable salt thereof, or composition is administered to the subject within 24 hours of birth, including, for example, within 5 minutes, 15 minutes, 30 minutes, 1 hour, 2 hours, 3 hours, 4 hours, 5 hours, six hours, 7 hours, 8 hours, 9 hours, 10 hours, 11 hours, 12 hours, or 18 hours of birth. In certain embodiments, the compound, pharmaceutically acceptable salt thereof, or composition is administered to the subject within 1 week of birth, including within 1 day, 2 days, 3 days, 4 days, 5 days, 6 days, or 7 days of birth. In certain embodiments, the compound, pharmaceutically acceptable salt thereof, or composition is administered to the subject within 1 month of birth, including within 1 week, 2 weeks, 3 weeks, or 4 weeks of birth.

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, and K1154Q. Barcia et al. (2012) Nat Genet. 44:1255-1260; Ishii et al. (2013) Gene 531:467-471; McTague et al. (2013) Brain. 136:1578-1591; Epi4K Consortium & Epilepsy Phenome/Genome Project. (2013) Nature 501:217-221; Lim et al. (2016) Neurogenetics; Ohba et al. (2015) Epilepsia 56: el21-el28; Zhou et al. (2018) Genes Brain Behav. e12456; Moller et al. (2015) Epilepsia. e114-20; Numis et al. (2018) Epilepsia. 1889-1898; Madaan et al. Brain Dev. 40(3): 229-232; McTague et al. (2018) Neurology. 90(1): e55-e66; Kawasaki et al. (2017) J Pediatr. 191:270-274; Kim et al. (2014) Cell Rep. 9(5): 1661-1672; Ohba et al. (2015) Epilepsia. 56(9): e121-8; Rizzo et al. (2016) Mol Cell Neurosci. 72:54-63; Zhang et al. (2017) Clin Genet. 91(5): 717-724; Mikati et al. (2015) Ann Neurol. 78(6): 995-9; Baumer et al. (2017) Neurology. 89(21): 2212; Dilena et al. (2018) Neurotherapeutics. 15(4): 1112-1126. These mutations may be gain-of-function, missense mutations that are dominant (i.e., present on only one allele) and result in change-in-function of the encoded potassium channel that causes a marked increase in whole cell current when tested in Xenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioral and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44:1188-1190). Functional studies of the mutated KCNT1 genes associated with ADNFLE indicated that the underlying mutations (M896I, R398Q, Y796H, and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56: el21-el28). Treatment targeting the KCNT1 channel suggests that these mutations are gain-of-function mutations (Fukuoka et al. (2017) Brain Dev 39:80-83).

In one aspect, disclosed herein is a method of treating treat a disorder associated with excessive neuronal excitability or a disorder associated with a gain-of-function mutation in a gene such as KCNT1 (for example, epilepsy and other encephalopathies (e.g., MMFSI or EIMFS), ADNFLE, West syndrome, infantile spasms, epileptic encephalopathy, focal epilepsy, Ohtahara syndrome, DEE, Lennox-Gastaut syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures), cardiac dysfunctions (e.g., cardiac arrhythmia, Brugada syndrome, myocardial infarction), pain and related conditions (e.g., neuropathic pain, acute/chronic pain, migraine, etc.), muscle disorders (e.g., myotonia, neuromyotonia, cramp muscle spasms, spasticity), itch and pruritis, ataxia and cerebellar ataxias, psychiatric disorders (e.g., major depression, anxiety, bipolar disorder, schizophrenia), learning disorders, Fragile X, neuronal plasticity, and autism spectrum disorders), comprising administering to a subject in need thereof a compound disclosed herein or a pharmaceutically acceptable salt thereof or a pharmaceutical composition disclosed herein.

In some examples, the subject presenting with a disorder that may be associated with a gain-of-function mutation in KCNT1 is genotyped to confirm the presence of a known gain-of-function mutation in KCNT1 prior to administration of the compounds or a pharmaceutically acceptable salt thereof or compositions disclosed herein. For example, whole exome sequencing can be performed on the subject. Gain-of-function mutations associated with EIMFS may include, but are not limited to, V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, and K1154Q. Gain-of-function mutations associated with ADNFLE may include, but are not limited to, M896I, R398Q, Y796H, R928C, and G288S. Gain-of-function mutations associated with West syndrome may include, but are not limited to, G652V and R474H. Gain-of-function mutations associated with temporal lobe epilepsy may include, but are not limited to, R133H and R565H. Gain-of-function mutations associated with Lennox-Gastaut may include, but are not limited to, R209C. Gain-of-function mutations associated with seizures may include, but are not limited to, A259D, G288S, R474C, and R474H. Gain-of-function mutations associated with leukodystrophy may include, but are not limited to, G288S and Q906H. Gain-of-function mutations associated with Multifocal Epilepsy may include, but are not limited to, V340M. Gain-of-function mutations associated with early-onset epilepsy (EOE) may include, but are not limited to, F346L and A934T. Gain-of-function mutations associated with Early-onset epileptic encephalopathies (EOEE) may include, but are not limited to, R428Q. Gain-of-function mutations associated with developmental and epileptic encephalopathies may include, but are not limited to, F346L, R474H, and A934T. Gain-of-function mutations associated with epileptic encephalopathies may include, but are not limited to, L437F, Y796H, P924L, and R961H. Gain-of-function mutations associated with Early Infantile Epileptic Encephalopathy (EIEE) may include, but are not limited to, M896K. Gain-of-function mutations associated with drug-resistant epilepsy and generalized tonic-clonic seizure may include, but are not limited to, F346L. Gain-of-function mutations associated with migrating partial seizures of infancy may include, but are not limited to, R428Q. Gain-of-function mutations associated with Leukoencephalopathy may include, but are not limited to, F932I. Gain-of-function mutations associated with NFLE may include, but are not limited to, A934T and R950Q. Gain-of-function mutations associated with Ohtahara syndrome may include, but are not limited to, A966T. Gain-of-function mutations associated with infantile spasms may include, but are not limited to, P924L. Gain-of-function mutations associated with Brugada Syndrome may include, but are not limited to, R1106Q. Gain-of-function mutations associated with Brugada Syndrome may include, but are not limited to, R474H.

In other examples, the subject is first genotyped to identify the presence of a mutation in KCNT1, and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1, as may be assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400%, or more. The subject can then be confirmed to have a disease or condition associated with a gain-of-function mutation in KCNT1.

In particular examples, the subject is confirmed as having a KCNT1 allele containing a gain-of-function mutation (e.g., V271F, G288S, R398Q, R428Q, R474Q, R474H, R474C, G652V, I760M, Y796H, M896I, P924L, R928C, or A934T).

The compounds or pharmaceutically acceptable salts thereof disclosed herein or the pharmaceutical composition disclosed herein (e.g., a pharmaceutical composition comprising a compound or pharmaceutically acceptable salt thereof disclosed herein, and a pharmaceutically acceptable excipient) can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the compounds or pharmaceutically acceptable salts thereof disclosed herein or the pharmaceutical compositions disclosed herein can be used to treat a subject with conditions associated with excessive neuronal excitability, for example, epilepsy and other encephalopathies (e.g., EIMFS, ADNFLE, West syndrome, infantile spasms, epileptic encephalopathy, focal epilepsy, Ohtahara syndrome, developmental and epileptic encephalopathy, Lennox-Gastaut syndrome, seizures) or cardiac dysfunctions (e.g., cardiac arrhythmia, Brugada syndrome, myocardial infarction), regardless of whether or not the disorder is associated with a gain-of-function mutation in KCNT1.

In some variations of the foregoing, a “subject” to which administration is contemplated includes, but is not limited to, humans (i.e., a male or female of any age group, e.g., a fetal subject (e.g., a subject that is in utero), a pediatric subject (e.g., a newborn (28 days or younger), an infant, child, adolescent) or an adult subject (e.g., a young adult, middle-aged adult, or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Some variations of the foregoing, “treating” or “treatment”, as used herein, contemplate an action that occurs while a subject is suffering from the specified disease, disorder or condition, which reduces the severity of the disease, disorder or condition, or retards or slows the progression of the disease, disorder or condition (also “therapeutic treatment”). In some variations, “treating” or “treatment” refers to a method or procedure for obtaining beneficial or desired results—for example, clinical results. Beneficial or desired results may include: (1) alleviating one or more symptoms caused by or associated with a disease, disorder, or condition; (2) reducing the extent of the disease, disorder, or condition; (3) slowing or stopping the development or progression of one or more symptoms caused by or associated with the disease, disorder, or condition (for example, stabilizing the disease, disorder, or condition); and (4) relieving the disease, for example, by causing the regression of one or more clinical symptoms (e.g., ameliorating the disease state, enhancing the effect of another medication, delaying or stopping the progression of the disease, increasing the quality of life, and/or prolonging survival rates).

In some variations of the foregoing, an “effective amount” of a compound or pharmaceutically acceptable salt thereof refers to an amount sufficient to elicit the desired biological response. As will be appreciated by those of ordinary skill in this art, the effective amount of a compound or pharmaceutically acceptable salt thereof may vary depending on such factors as the desired biological endpoint, the pharmacokinetics of the compound or pharmaceutically acceptable salt thereof, the disease being treated, the mode of administration, and the age, weight, health, and condition of the subject.

In some embodiments, a therapeutically effective amount of the compound or pharmaceutically acceptable salt thereof disclosed herein is administered to the subject (e.g., a human). In some variations of the foregoing, a “therapeutically effective amount” of a compound or pharmaceutically acceptable salt thereof is an amount sufficient to provide a therapeutic benefit in the treatment of a disease, disorder or condition, or to delay or minimize one or more symptoms associated with the disease, disorder or condition. A therapeutically effective amount of a compound or pharmaceutically acceptable salt thereof means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of the disease or condition, or enhances the therapeutic efficacy of another therapeutic agent.

In some embodiments, the method provided involves treating a disorder associated with a gain-of-function mutation of KCNT1. In some variations, a “disorder associated with a gain-of-function mutation in KCNT1” refers to a disorder that is associated with, is partially or completely caused by, or has one or more symptoms that are partially or completely caused by, a mutation in KCNT1 that results in a gain-of-function phenotype, i.e., an increase in activity of the potassium channel encoded by KCNT1 resulting in an increase in whole cell current. In some variations, a “gain-of-function mutation of KCNT1” is a mutation in KCNT1 that results in an increase in activity of the potassium channel encoded by KCNT1. Activity can be assessed by, for example, ion flux assay or electrophysiology (e.g., using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400%, or more compared to the activity of a potassium channel encoded by a wild-type KCNT1.

### IV. Pharmaceutical Compositions and Routes of Administration

Compounds or pharmaceutically acceptable salts thereof provided in accordance with the present invention are usually administered in the form of pharmaceutical compositions. Therefore, disclosed herein are pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions may be prepared in a manner disclosed in the pharmaceutical art, including, for example, in Remington's Pharmaceutical Sciences, Mace Publishing Co., Philadelphia, Pa. 17th Ed. (1985) and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions disclosed herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersions, and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound or pharmaceutically acceptable salt thereof as disclosed herein in the required amount in the appropriate solvent with various other ingredients as enumerated above, as desired, followed by filtered sterilization. Generally, dispersions are prepared by incorporating the various sterilized active ingredients into a sterile vehicle which contains the basic dispersion medium and the desired other ingredients from those enumerated above. In the case of sterile powders for the preparation of sterile injectable solutions, exemplary methods of preparation include vacuum-drying and freeze-drying techniques which yield a powder of the active ingredient plus any additional desired ingredient from a previously sterile-filtered solution thereof.

Oral administration is another route for administration of the compounds or pharmaceutically acceptable salts thereof as disclosed herein. Administration may be via capsule or enteric coated tablets, or the like. In making the pharmaceutical compositions that include at least one compound or pharmaceutically acceptable salt thereof described herein, the active ingredient may be diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. In certain embodiments, the compositions disclosed herein can additionally include lubricating agents such as talc, magnesium stearate, and mineral oil; wetting agents; emulsifying and suspending agents; preserving agents such as methyl and propylhydroxy-benzoates; sweetening agents; glidants; and flavoring agents.

The compositions disclosed herein can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another embodiment for use in the methods disclosed herein may employ transdermal delivery devices (“patches”). Such transdermal patches may be used to provide continuous or discontinuous infusion of the compounds or pharmaceutically acceptable salts thereof as disclosed herein in controlled amounts. The construction and use of transdermal patches for the delivery of pharmaceutical agents is described, for example, in U.S. Pat. Nos. 5,023,252, 4,992,445 and 5,001,139. Such patches may be constructed for continuous, pulsatile, or on-demand delivery of pharmaceutical agents.

The compositions disclosed herein may be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from about 1 mg to about 2 g of a compound or pharmaceutically acceptable salt thereof as described herein, and for parenteral administration, preferably from about 0.1 to about 700 mg of a compound or pharmaceutically acceptable salt thereof as described herein. It will be understood, however, that the amount of the compound or pharmaceutically acceptable salt thereof actually administered usually will be determined by a physician, in the light of the relevant circumstances, including the condition to be treated, the chosen route of administration, the actual compound or pharmaceutically acceptable salt thereof administered and its relative activity, the age, weight, and response of the individual patient, the severity of the patient's symptoms, and the like.

For preparing solid compositions such as tablets, the principal active ingredient may be mixed with a pharmaceutical excipient to form a solid preformulation composition containing a homogeneous mixture of a compound or pharmaceutically acceptable salt thereof as disclosed herein. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills disclosed herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. In certain embodiments, the compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a facemask tent or intermittent positive pressure breathing machine. Solution, suspension, or powder compositions may be administered, such as orally or nasally, from devices that deliver the formulation in an appropriate manner.

In some embodiments, there is provided a pharmaceutical composition comprising a compound, or pharmaceutically acceptable salt thereof, as disclosed herein and at least one pharmaceutically acceptable excipient and/or carrier.

Those skilled in the art will recognize or be able to ascertain using no more than routine experimentation many equivalents to the specific embodiments described herein. The scope of the present embodiments described herein is not intended to be limited to the above Description or the Examples that follow, but rather is as set forth in the appended claims. Those of ordinary skill in the art will appreciate that various changes and modifications to this description may be made without departing from the spirit or scope of the embodiments disclosed herein, as defined in the claims.

### EXAMPLES

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using general methods and procedures. For example, methods of making a compound of Formula (I), including the compound of Formula (I-A) are described in in WO 2020/227101, which is incorporated herein by reference in its entirety. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are described in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high performance liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods described in the art.

Epilepsy of infancy with migrating focal seizures (EIMFS) is an epileptic encephalopathy associated with highly intractable seizures and devastating neurodevelopmental disability. EIMFS has been associated with heterozygous gain-of-function variants in the Na-activated K channel Slack (KNa1.1), which is encoded by KCNT1. Previously available Slack/KNa1.1 antagonists have been limited in clinical use due to poor specificity and a small therapeutic window. A specific KNa1.1 antagonist was recently discovered and shown to suppress spikes and seizures in a homozygous mouse model of KCNT1 epilepsy (Griffin et al., ACS Med Chem Lett. 2021). Human KCNT1 epilepsy is a heterozygous condition, however, so the effects of this compound were evaluated on an epileptic mouse model heterozygous for the mutation Kcnt1^{R455H/+}. Wu et al. PMID 36824888 and Quraishi et al PMID 32081855. While not wishing to be bound by theory, it is believed that the R474H gain-of-function mutation in humans is equivalent to the R455H mutation in mice. Kcnt1^{R455H/+} mice are known to have frequent spontaneous interictal epileptiform discharges (spikes). Infrequent spontaneous seizures and decreased thresholds for induced seizures also occur in some Kcnt1^{R455H/+} mice. In the Examples that follow, the effects of a Slack inhibitor on spontaneous epileptiform activity was tested, as well as the effects of a Slack inhibitor on acute seizures provoked by pentylenetetrazole (PTZ) and the effects of a Slack inhibitor on survival of R455H homozygous mice (Kcnt1^{R455H/R455H}).

### Example 1—Effect of Compound of Formula (I-A) in Epilepsy Mouse Model Having a Heterozygous Kcnt1^{R455H/+} Mutation in PTZ-Induced Seizures

Kcnt1^{R455H/+} heterozygous mice have increased sensitivity to PTZ seizure induction as compared with wild-type littermates. It was determined herein whether a compound of Formula (I-A) delivered prior to PTZ could delay the onset of convulsive seizures. Formula (I-A) (75 mg/kg s.c.) or a vehicle control was administered as a single dose. After a pretreatment period of one or two hours, mice were administered PTZ at a dose of 55 mg/kg for induction of seizures. Seizures were then scored based on behavior (no EEG). The latency to convulsive seizures was compared between groups to determine if the compound can prevent induced seizures.

Animals: Adult Kcnt1^{R455H/+} heterozygous C57/B6J mice were used in this example. Mice were approximately 2 months old. No preference was made for male or female animals.

Drug preparation and delivery: A 7.5 mg/mL solution of a compound of Formula (I-A) was prepared on each day of injection in 10% DMSO/10% Solutol/80% deionized water with a volume sufficient for 10-20 animals. After at least 30 minutes of acclimatization to the testing environment, either the drug or vehicle control was delivered to each animal subcutaneously at a dose of 75 mg/kg based on current body weight.

PTZ injection and testing: Kcnt1^{R455H/+} mice were tested with PTZ 55 mg/kg IP administered 1 or 2 hours after administration of either the compound of Formula (I-A) 75 mg/kg SC or vehicle control. Mice were then monitored continuously for 30 min by an observer blinded to treatment group. Mice were observed for the presence or absence of generalized clonic seizures with loss of posture. Latency to first clonic seizure with loss of posture was recorded and compared using the time to event analysis; log-rank Mantel-Cox in GraphPad Prism, p<0.05 was considered statistically significant. Seizures induced by PTZ were scored using a modified Racine scale, and the latency to convulsive seizures was compared between drug and vehicle groups (log rank test).

Behavior was scored live, and mice were recorded on video for later confirmation. Seizure severity scores were based on a recent report that is specifically designed for mouse PTZ experiments (Van Erum et al., Epilepsy and Behavior 2019 June; 95:51-55). Behavioral seizure scores were as follows:
−1: normal baseline
0: whisker trembling
1: behavioral arrest
2: facial jerking
3: neck jerks/myoclonus
4: clonic (sitting)
5: clonic, tonic-clonic (lying on belly)
6: clonic, tonic-clonic (lying on side), and wild jumping
7: tonic extension, possibly leading to respiratory arrest and death.

The time at which mice reached each stage was recorded. It was difficult to reliably ascertain grade 0-2 seizures without the aid of EEG, and there was inconsistency between reviewers for stage 3-5 seizures. As the clearest and most consistent (between examiners) scores were for stage 6-7, only these were used for reporting and analysis, although earlier scores were also recorded and saved. Latency to stage 6 or above seizures were plotted as survival curves, and differences were assessed with a long rank test. Scoring was cut off after 22 minutes post-PTZ administration (1320 seconds).

Latency to Stage 6+ seizures with 1-hour and 2-hour pretreatment: Mice were given 75 mg/kg of Formula (I-A) or a vehicle control subcutaneously 1 hour prior to PTZ 55 mg/kg IP injection. PTZ experimental mice were not implanted with EEGs so seizure behavior was scored live and confirmed by video. Mice were observed for the presence or absence of clonic or tonic seizures with loss of posture. Following 1 hour of pretreatment, 1/16 (6%) animals delivered the compound of Formula (I-A) had PTZ-induced convulsive seizures, as compared with 6/15 (40%) animals given the vehicle control (p=0.037, Fisher's exact test). Seizure progression: p=0.40 (two-way repeated measures ANOVA). Convulsive seizure latency: hazard ratio (drug/vehicle)=0.13 (95% CI 0.03-0.58), p=0.025 (log rank test).

Based on the EEG findings that suggested the drug was prolonged over many hours, and because the uptake time into brain was unknown, the experiment was repeated with drug or vehicle control given after a longer interval 2 hours prior to PTZ. Following 2 hours of pretreatment, 13 out of 20 (65%) mice that were given vehicle 2 hours prior to PTZ had a seizure that reached stage 6-7, whereas only 1 out of 20 (5%) mice that were given drug reached stage 6-7 (p=0.0001, Fisher exact test). Seizure progression: p=0.0020 (two-way repeated measures ANOVA)

Convulsive seizure latency: hazard ratio (drug/vehicle)=0.056 (95% CI 0.019-0.161), p=0.0001 (log rank test).

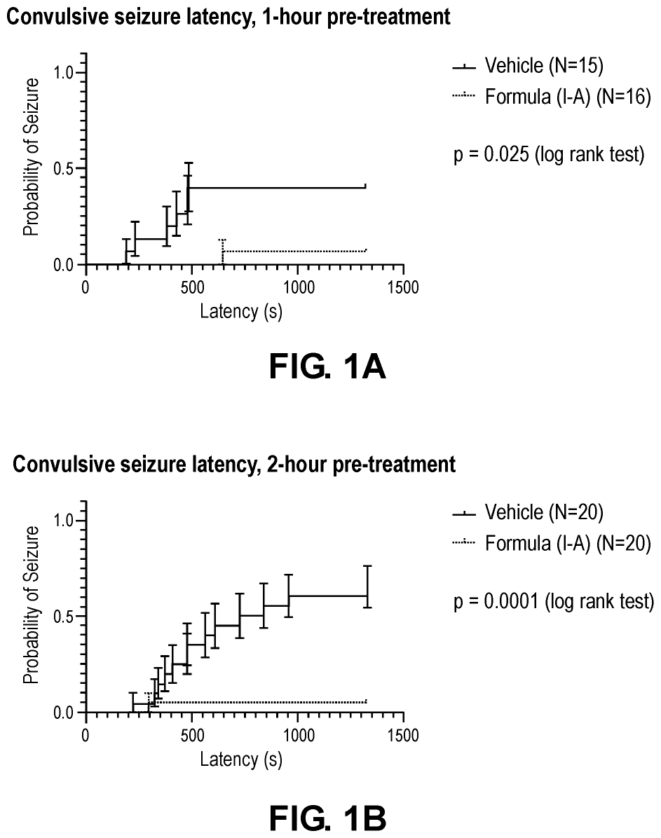

FIG. 1A shows the latency to convulsive seizures following PTZ administration when mice were given Formula (I-A) 75 mg/kg subcutaneously 1 hour prior to PTZ, and FIG. 1B shows the latency to convulsive seizures following PTZ administration when mice were given Formula (I-A) 75 mg/kg subcutaneously 2 hours prior to PTZ. These results confirm that Formula (I-A) was anticonvulsant against PTZ-induced seizures in Kcnt1^{R455H+} heterozygous mice.

The data for FIGS. 1A and 1B are shown below in Table 1 and 2, respectively.

TABLE 1
Latency to clonic or tonic seizures with loss of posture when
vehicle or Formula (I-A) administered 1 hour prior to PTZ

| | |
|---|---|
| Vehicle (N = 15) | Formula (I-A) (N = 16) |

| Animal ID | Latency (s) | Animal ID | Latency (s) |
|---|---|---|---|
| 790 | 234 | 783 | 1320 |
| 782 | 1320 | 775 | 1320 |
| 796 | 1320 | 786 | 1320 |
| 792 | 383 | 793 | 1320 |
| 785 | 1320 | 789 | 1320 |
| 802 | 1320 | 795 | 1320 |
| 804 | 1320 | 801 | 1320 |
| 806 | 1320 | 803 | 1320 |
| 808 | 486 | 805 | 1320 |
| 821 | 1320 | 807 | 1320 |
| 797 | 190 | 820 | 1320 |
| 940 | 1320 | 799 | 190 |
| 984 | 1320 | 986 | 1320 |
| 35 | 481 | 983 | 1320 |
| 33 | 428 | 996 | 1320 |
| | | 993 | 647 |

TABLE 2
Latency to clonic or tonic seizures with loss of posture when
vehicle or Formula (I-A) administered 2 hours prior to PTZ

| | |
|---|---|
| Vehicle (N = 20) | Formula (I-A) (N = 20) |

| Animal ID | Latency (s) | Animal ID | Latency (s) |
|---|---|---|---|
| 37 | 562 | 91 | 1320 |
| 62 | 475 | 61 | 1320 |
| 59 | 1320 | 54 | 1320 |
| 58 | 1320 | 60 | 1320 |
| 13 | 341 | 26 | 1320 |
| 10 | 1320 | 16 | 294 |
| 21 | 1320 | 9 | 1320 |
| 19 | 373 | 20 | 1320 |
| 191 | 407 | 193 | 1320 |
| 198 | 326 | 199 | 1320 |
| 181 | 835 | 187 | 1320 |
| 179 | 1320 | 182 | 1320 |
| 185 | 1320 | 184 | 1320 |
| 167 | 1320 | 188 | 1320 |
| 178 | 606 | 183 | 1320 |
| 168 | 1320 | 180 | 1320 |
| 243 | 223 | 249 | 1320 |
| 248 | 952 | 244 | 1320 |
| 176 | 721 | 245 | 1320 |
| 175 | 477 | 173 | 1320 |

In the data above, latency was scored as 1320 seconds if no seizure was observed over the duration of the experiment.

Treatment with 75 mg/kg Formula (I-A) significantly increased the latency to and reduced the incidence of PTZ-induced seizures in Kcnt1^{R455H/+} heterozygous mice compared to vehicle-treated mice.

Latency to seizures was also prolonged by the drug (p=0.025 for 1 hour pre-treatment, p=0.0001 for 2-hour pretreatment). Baseline epileptiform spike rates on EEG were variable between animals, but there was a marked per-animal reduction in the 24 hours following administration of the compound of Formula (I-A) (p=0.005, N=12). These results suggest that KNa1.1 antagonists, like the compound of Formula I-A, may be an effective treatment for KCNT1 epilepsy.

### Example 2—Effects of Formula (I-A) on Spontaneous Epileptiforn Activity and EEG

Kcnt1R455H/+heterozygous mice exhibit frequent spontaneous interictal epileptiform discharges (spikes). Infrequent spontaneous seizures also occur in some Kcnt1^{R455H/+} heterozygous mice. This experiment sought to determine if administration of a compound of Formula (I-A) could reduce interictal spikes.

Animals: Adult Kcnt1^{R455H/+} heterozygous mice on a C57/B6J background were used in all experiments disclosed in this example. Mice were approximately 2-3 months old. No preference was made for male or female animals. This strain is almost always stillborn in the homozygous condition. Seizures occur in some but not all heterozygotes, and interictal discharges occurs reliably.

Implantation of EEG electrodes: Mice were anesthetized with inhaled isoflurane 3-4% induction followed by approximately 1-1.5% maintenance titrated to effect. The head was fixed in a Kopf stereotactic apparatus, and the scalp was sterilized with povidone-iodine. The temperature was regulated to 37 degrees Celsius with a heating pad and rectal probe. Pre-emptive analgesia was provided with subgaleal bupivacaine 0.1% administered to form a wheal on the scalp, no more than 0.08 mL/10 g body weight. A linear midline scalp incision was made from behind the eyes to expose skull markers. Four holes were drilled (#60 drill bit): two anterior to the coronal suture (for left and right active recording), and two ⅔ of the way from the frontal to lambdoid suture (for reference and ground), approximately 8 mm from midline. Screw electrodes were inserted in each burr hole (size W00-96×1.6 mm soldered to 15 mm insulated wire) and connected to a plastic pedestal (PlasticsOne E363). This assembly was then secured with dental cement. Meloxicam 5 mg/kg IP was given for post-operative analgesia.

Drug preparation for mice under EEG: A 7.5 mg/mL solution of a compound of Formula (I-A) was prepared on the day of injection in 10% DMSO/10% Solutol/80% deionized water. After at least 72 hours of baseline recording, the compound of Formula (I-A) was delivered subcutaneously at a dose of 75 mg/kg based on pre-surgical body weight.

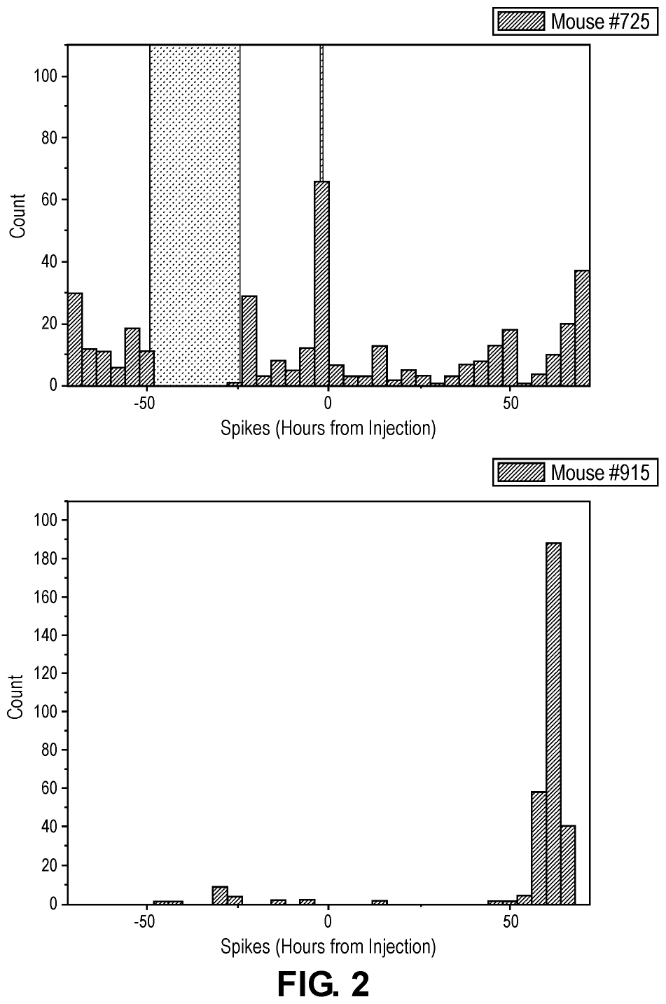

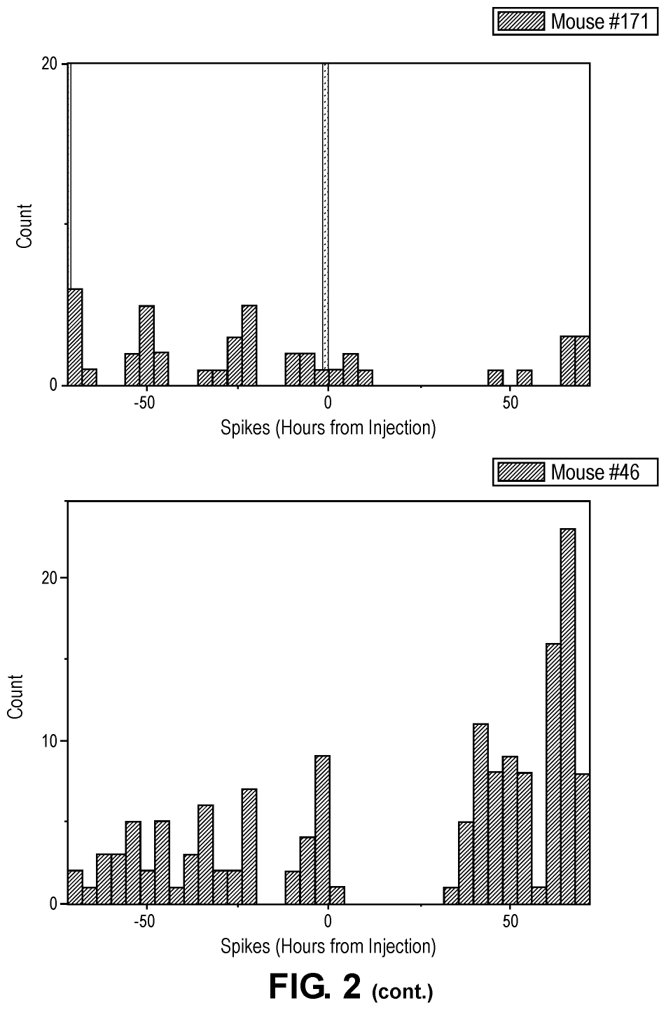

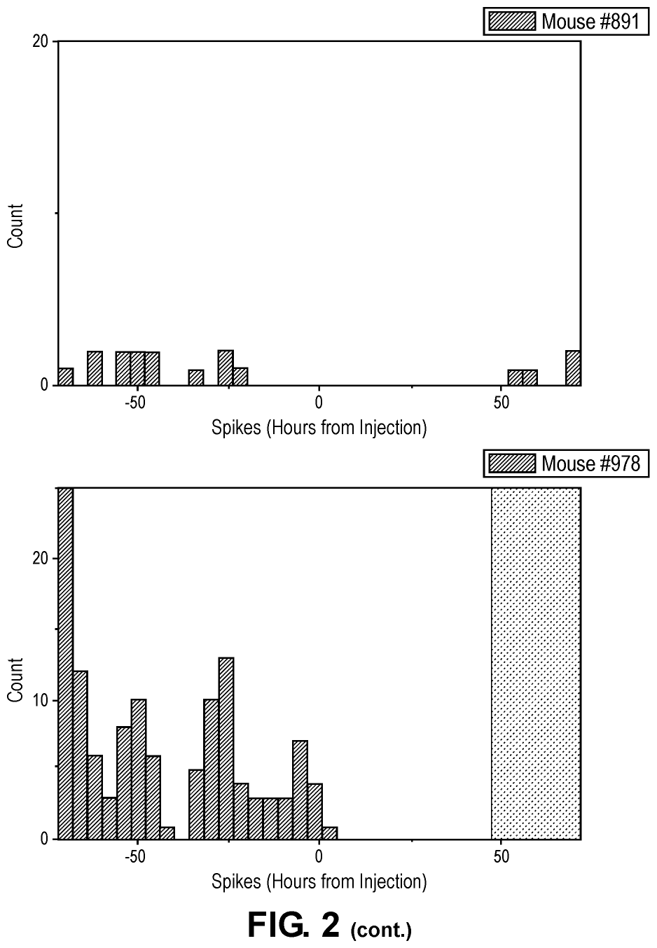

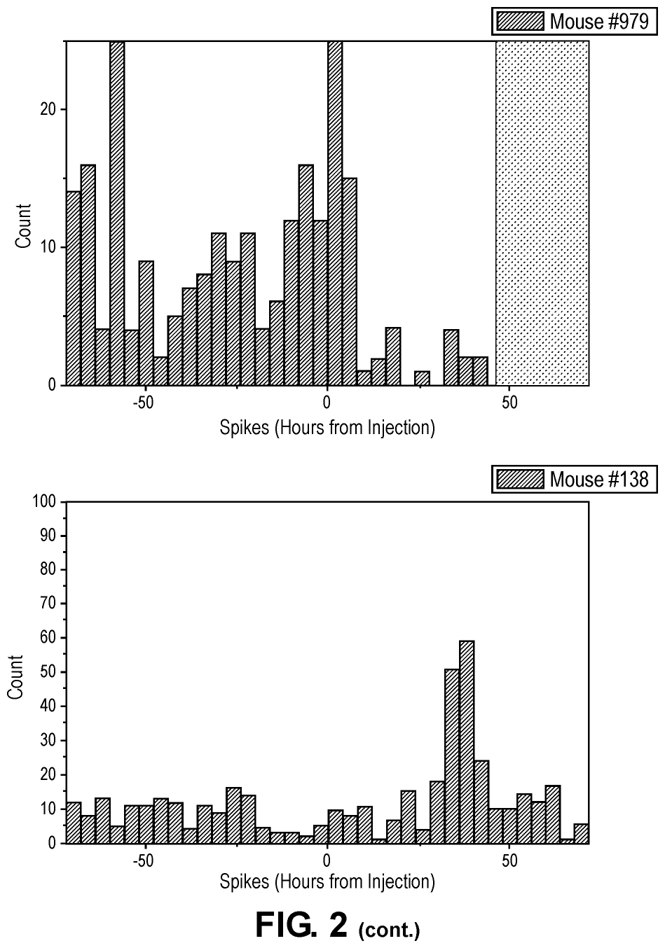

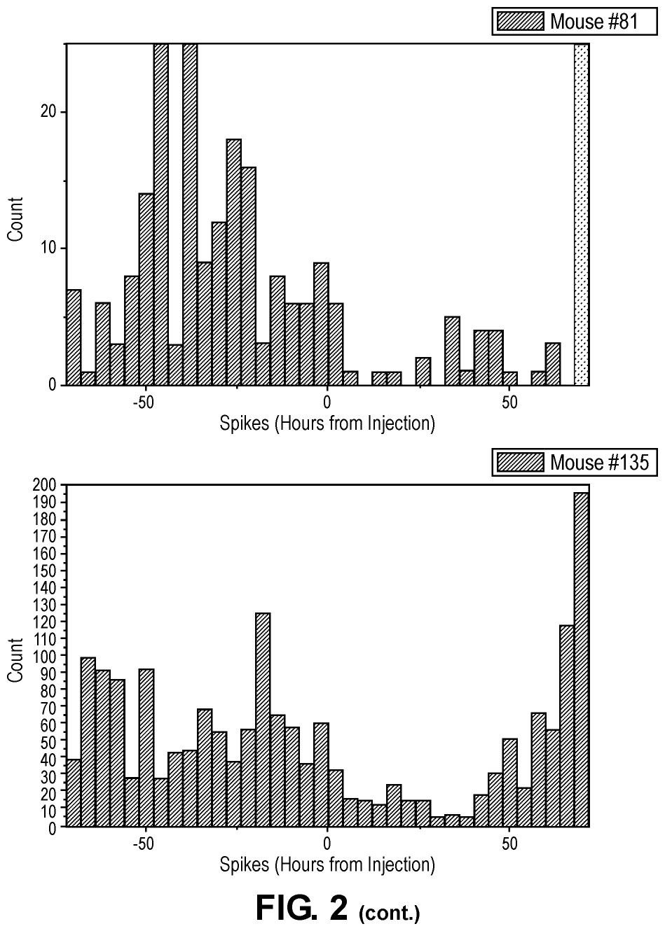

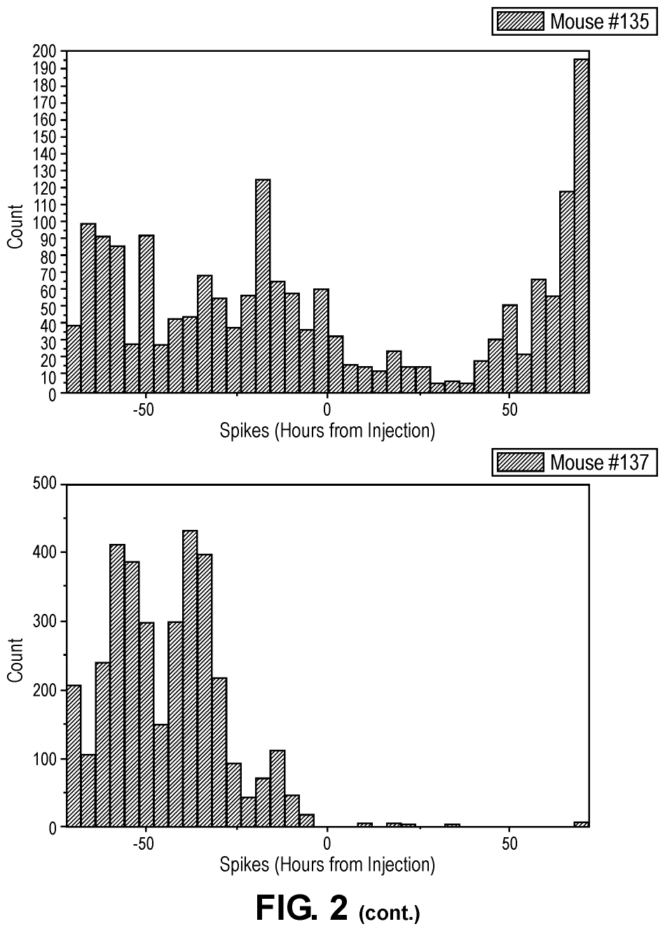

EEG recording and analysis: Mice were held in a heated cage for at least 24 hours to recover before connection to the EEG recording system. Two-channel EEG with video was recorded using a Compumedics Grael EEG amplifier at 512 Hz sampling rate. The entire EEG was reviewed manually, and all interictal discharges and seizures were identified and marked. Video recordings were used to confirm epileptiform activity. Mice that had adequate recording quality and interictal spikes were selected for drug delivery. EEG was marked for an additional 72 hours after injection of either vehicle or 75 mg/kg of Formula (I-A), after which mice were anesthetized. EEG recordings were manually scored by a reviewer blinded to treatment group. All EEG markings were reviewed and confirmed by a second reviewer. Interictal spike rates were averaged in each time period (baseline and the first, second, and third post-injection days), accounting where necessary for segments of missing data. Average discharge rates per mouse from the first 24 hours after injection were compared with rate from the 72-hour baseline with a two-tailed paired t-test. FIG. 2 shows the interictal spike rate observed for each of 12 individual mice. As shown in FIG. 2, interictal discharges were binned in 4-hour intervals and displayed over a 6-day period (i.e., 3 days before drug injection and 3 days after drug injection). Dotted segments indicate periods in which EEG data was not available. It was observed that some animals showed a change in morphology of interictal spikes following drug injection.

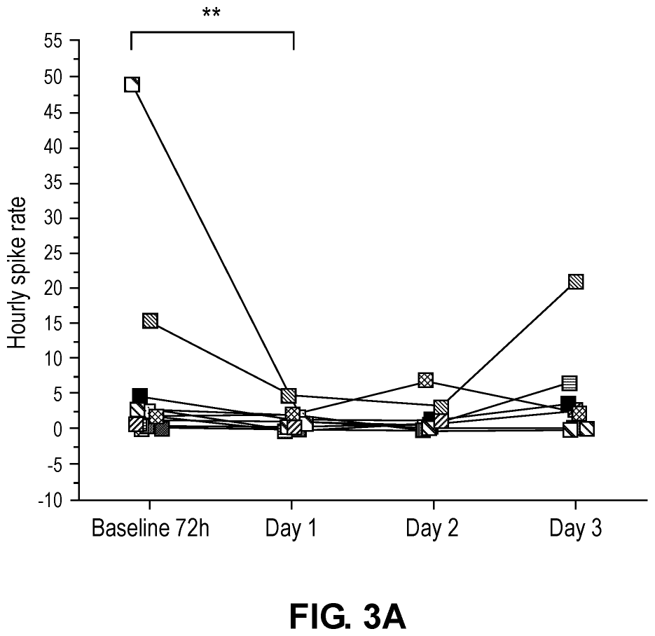

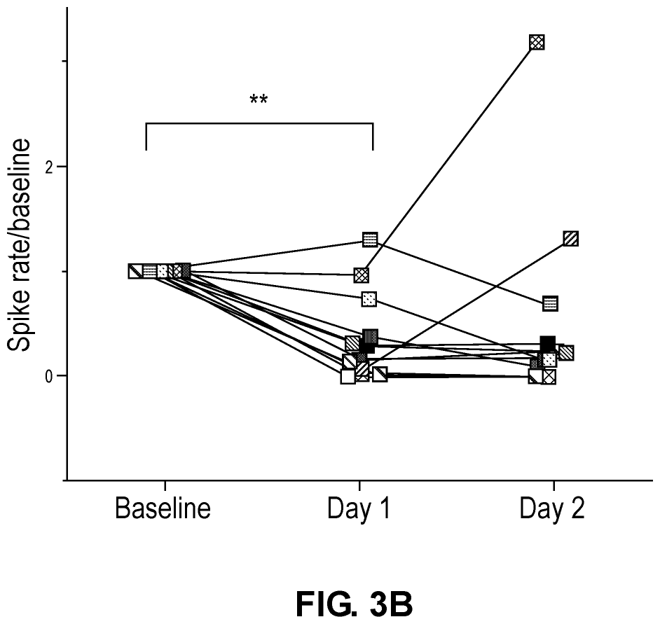

EEG data were collected and reviewed in Compumedics Profusion. Interictal spikes were marked from 72 hours before and after subcutaneous drug injection. Spike times were binned and summarized in Origin 2021b. Summary statistics for reduction in spikes each day following injection were compared with the baseline using a non-parametric test due to skew in the baseline spike rates. Average interictal discharge rates were determined in the baseline period and in the first, second, and third post-injection days (FIG. 3A). Average discharge rates per mouse from the first 24 hours after injection were compared with the rate from the 72-hour baseline with a two-tailed paired t-test (p=0.005) (Wilcoxon matched rank pairs test).

As shown in FIG. 3A, spike rates were reduced in the first 24 hours after dosing with Formula (I-A) in comparison to the preceding 72 hours (p=0.005, Wilcoxon matched-pairs signed rank test, N=12). The reduction persisted through the second day (p=0.042 vs. baseline) and was no longer evident in the third day (p>0.999), consistent with the pharmacokinetic profile of the Formula (I-A) compound. The Formula (I-A) compound-induced decrease in daily mean spike rate is more obvious when visualizing the baseline-adjusted spike rates (FIG. 3B). As shown in FIG. 3B, 11 out of 12 mice had a decrease in spike rates from baseline to Day 1.

### Example 3—Effects of a Compound of Formula (I-A) on Neuronal Electrophysiology

Cortical neurons from Kcnt1^{R455H} heterozygous mice were cultured and treated for 20 minutes with 10 μM of a compound of Formula (I-A) in DMSO. Presumed glutamatergic neurons were identified based on pyramidal morphology. Presumed GABAergic neurons were identified based on spindle morphology (and fast spiking pattern in the control recordings). Current clamp and voltage clamp recordings were performed on an Axon Multiclamp 700B amplifier. It was observed that pyramidal cells treated with vehicle control had many action potentials and mild adaptation in response to an inward current and large outward currents with depolarization. Pyramidal cells treated with Formula (I-A) had less sustained action potential firing and diminished outward currents. Presumed GABAergic neurons treated with vehicle control had fast spiking without adaptation and large outward currents, while presumed GABAergic neurons treated with Formula (I-A) had markedly reduced spike rates and outward currents.

### Example 4—Effects of a Compound of Formula (I-A) on Survival of R455H Mice Homozygotes

Although KCNT1^{R455H/+} heterozygous mice have not been noted to exhibit premature mortality, homozygous littermates (KCNT1^{R455H/R455H}) are described in the art as being stillborn or dying within the first day following birth, despite the embryos appearing viable in utero. Quraishi et. al. PMID 32081855. Without wishing to be bound by theory, it is thought that inhibition of mutant channel activity by KCNT1 inhibitors, such as the compound of Formula (I-A), during gestation might aid the survival of homozygous pups. Timed matings were performed on heterozygote breeding pairs, and a compound of Formula (I-A) was administered to the pregnant dams. No dosing of the live pups with Formula (I-A) was performed. All surviving and stillborn offspring that could be recovered were then genotyped, and brains were dissected and sent for measurement of Formula (I-A). The presence of any surviving homozygotes would suggest efficacy for Formula (I-A).

Breeding and dosing: Timed matings were performed on three KCNT1^{R455H+}×KCNT1^{R455H/+} breeding pairs. Maternal injections of Formula (I-A) (75 mg/kg s.c.) were performed for four pregnant dams from three Kcnt1^{R455H/+}×Kcnt1^{R455H/+} breeding pairs and one Kcnt1^{+/+}×Kcnt1^{+/+} breeding pair. Starting approximately 13 days after the mating plug was identified, pregnant dams were administered 75 mg/kg Formula (I-A) (s.c.) every 3 days (as permitted) through the time of delivery. There was some variability in the days of administration as noted. Offspring were observed daily after delivery. The records of litters and treatment dates are shown below in Table 3. In round one, two doses of Formula (I-A) were delivered to each of the two dams from the Kcnt1^{R455H/+}×Kcnt1^{R455H/+} crosses. From these matings, it was surprisingly found that four Kcnt1^{R455H/R455H} pups were born alive with three surviving for one day and one surviving two days, as shown below in Table 4. In round two, three doses of Formula (I-A) were delivered to one dam from a Kcnt1^{R455H/+}×Kcnt1^{R455H/+} cross. This cross resulted in the birth of one Kcnt1^{R455H/R455H} pup, which surprisingly survived for four days (Table 4).

TABLE 3
Record of Litters and Treatment Dates

| Litter # | 1 | 2 | 3 | 4 |
|---|---|---|---|---|
| Round # | 1 | 1 | 2 | 2 |
| Female (Dam) | 901 | 814 | 954 | 952 |
| ID | | | | |
| Female | Kcnt1^{R455H/+} | Kcnt1^{R455H/+} | Kcnt1^{R455H/+} | Kcnt1^{R455H/+} |
| genotype | | | | |
| Male genotype | Kcnt1^{R455H/+} | Kcnt1^{R455H/+} | Kcnt1^{R455H/+} | Kcnt1^{R455H/+} |
| Delivery date | Day 0 | Day 2 | Day 29 | Day 31 |
| Plug date | Day −19 | Day −17 | Day 10 | Day 11 |
| Injection 1 date | Day −5 | Day −4 | Day 22 | Day 23 |
| Injection 1 | 75 | 75 | 75 | 75 |
| dose (mg/kg) | | | | |
| Injection 1 | 15 | 14 | 13 | 13 |
| day # | | | | |
| Injection 2 date | Day −3 | Day −1 | Day 25 | Day 26 |
| Injection 2 | 75 | 75 | 75 | 75 |
| dose (mg/kg) | | | | |
| Injection 2 | 17 | 17 | 16 | 16 |
| day # | | | | |
| Injection 3 date | N/A | N/A | Day 28 | Day 29 |
| Injection 3 | N/A | N/A | 75 | 75 |
| dose (mg/kg) | | | | |
| Injection 3 | N/A | N/A | 19 | 19 |
| day # | | | | |

TABLE 4
List of pups identified for 4 litters

| | | | | | Age survived |
|---|---|---|---|---|---|
| Pup | Litter | | | | before death or |
| # | # | Genotype | DOB | DOD | euthanasia (days) |
| 1 | 1 | Kcnt1^{R455H/+} | Day 0 | Day 14 | 14 |
| 2 | 1 | Kcnt1^{+/+} | Day 0 | Day 14 | 14 |
| 3 | 1 | Kcnt1^{+/+} | Day 0 | Day 14 | 14 |
| 4 | 1 | Kcnt1^{R455H/+} | Day 0 | Day 14 | 14 |
| 5 | 1 | Kcnt1^{R455H/+} | Day 0 | Day 14 | 14 |
| 6 | 1 | Kcnt1^{R455H/R455H} | Day 0 | Day 1 | 1 |
| 7 | 1 | Kcnt1^{R455H/R455H} | Day 0 | Day 1 | 1 |
| 8 | 2 | Kcnt1^{R455H/+} | Day 2 | Day 16 | 14 |
| 9 | 2 | Kcnt1^{R455H/+} | Day 2 | Day 16 | 14 |
| 10 | 2 | Kcnt1^{R455H/+} | Day 2 | Day 16 | 14 |
| 11 | 2 | Kcnt1^{R455H/+} | Day 2 | Day 16 | 14 |
| 12 | 2 | Kcnt1^{R455H/+} | Day 2 | Day 16 | 14 |
| 13 | 2 | Kcnt1^{R455H/R455H} | Day 2 | Day 4 | 1 |
| 14 | 2 | Kcnt1^{R455H/R455H} | Day 2 | Day 4 | 2 |
| 15 | 2 | N/A | Day 2 | N/A | N/A |
| 16 | 3 | Kcnt1^{R455II/+} | Day 29 | Day 43 | 14 |
| 17 | 3 | Kcnt1^{+/+} | Day 29 | Day 43 | 14 |
| 18 | 3 | Kcnt1^{R455H/+} | Day 29 | Day 43 | 14 |
| 19 | 3 | Kcnt1^{+/+} | Day 29 | Day 43 | 14 |
| 20 | 3 | Kcnt1^{+/+} | Day 29 | Day 43 | 14 |
| 21 | 3 | Kcnt1^{R455H/+} | Day 29 | Day 43 | 14 |
| 22 | 3 | Kcnt1^{+/+} | Day 29 | Day 43 | 14 |
| 23 | 3 | Kcnt1^{R455H/R455H} | Day 29 | Day 33 | 4 |
| 24 | 4 | Kcnt1^{+/+} | Day 31 | Day 45 | 14 |
| 25 | 4 | Kcnt1^{+/+} | Day 31 | Day 45 | 14 |
| 26 | 4 | Kcnt1^{+/+} | Day 31 | Day 45 | 14 |
| 27 | 4 | Kcnt1^{+/+} | Day 31 | Day 45 | 14 |
| 28 | 4 | Kcnt1^{+/+} | Day 31 | Day 45 | 14 |
| 29 | 4 | Kcnt1^{+/+} | Day 31 | Day 45 | 14 |
| 30 | 4 | Kcnt1^{+/+} | Day 31 | Day 45 | 14 |
| 31 | 4 | Kcnt1^{+/+} | Day 31 | Day 45 | 14 |

Whole brains were collected from all pups at 14 days after birth or at the time they are found deceased. Tails were genotyped through an automated system (Transnetyx) which was previously validated against PCR-based RFLP and sequencing. The whole brains were analyzed to determine brain exposure of Formula (I-A).

Bioanalysis of Brain Samples: Brain tissue was homogenized in 10:1 (v/wt) ratio using 10 mM PBS in methanol (2:1, v/v). 20 μL of samples were then precipitated with 200 μL IS solution (100 ng/mL Labetalol & 100 ng/mL Tolbutamide in ACN). The mixture was vortex-mixed and centrifuged at 4000 rpm for 15 min, 4° C. An aliquot of 100 μL supernatant was transferred to a sample plate and mixed with 100 μL water. The plate was shaken at 800 rpm for 10 minutes. 1 μL supernatant was then injected for LC-MS/MS analysis. A 1-3000 ng/ml PRX-0002904 calibration curve was prepared in mouse brain homogenate. Ultra-performance liquid chromatography (UPLC) parameters conditions are provided in Table 5 below, and the results are shown in Table 6 below.

TABLE 5
UPLC Parameters and Conditions

| Mobile Phase A | 0.1% FA in water | |
|---|---|---|
| Mobile Phase B | 0.1% FA in CAN | |
| Column | Waters ACQUITY UPLC BEH C18 | |
| | 2.1 × 50 mm, 1.7 μm | |
| Flow Rate | 0.6 | ml/min |
| Retention Time (Formula (I-A)) | 1.22 | min |
| Retention Time (tolbutamide) | 0.69 | min |

| Time (min) | Mobile Phase B (%) |
|---|---|
| Initial | 50 |
| 0.10 | 50 |
| 1.00 | 98 |
| 1.50 | 98 |
| 1.55 | 50 |
| 2.50 | Stop |

TABLE 6
Formula (I-A) Brain Exposure in KCNT1^{R455H/R455H} Pups

| Mouse | Homogenization | Brain Homogenate | Final Brain |
|---|---|---|---|
| # | Ratio | Concentration (ng/mL) | nitrationation (ng/g) |
| 6 | 10 | 15.2 | 167 |
| 7 | 10 | 133 | 1458 |
| 13 | 10 | 109 | 1200 |
| 14 | 20 | 16.9 | 354 |
| 23 | 10 | BLQ | BLQ |

The results of this study suggest that treatment of pregnant mice with a compound of Formula (I-A) results in Formula (I-A) exposure in fetal Kcnt1^{R455H/R455H} brain and protection Kcnt1^{R455H/R455H} pups from immediate lethality during or following birth. Two of five Kcnt1^{R455H/R455H} pups survived multiple days, as shown in Table 4 above.

1. A method of treating a neurological disorder, a disorder associated with excessive neuronal excitability, or a disorder associated with a gain-of-function mutation of a gene, wherein the method comprises administering to a subject in need thereof an effective amount of a compound of Formula (I-A): 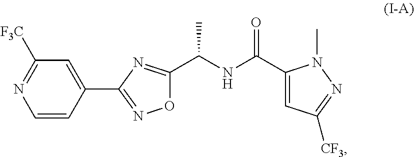 [SMILES: C[C@H](NC(=O)c1cc(C(F)(F)F)nn1C)c1nc(-c2ccnc(C(F)(F)F)c2)no1]
or a pharmaceutically acceptable salt thereof,
wherein the subject is in utero and the compound of Formula (I-A) or a pharmaceutically acceptable salt thereof is administered to a pregnant mother of the subject.

2. The method of claim 1, wherein the disorder is a disorder associated with a gain-of-function mutation of KCNT1.

3. The method of claim 1, wherein the neurological disorder, the disorder associated with excessive neuronal excitability, or the disorder associated with a gain-of-function mutation of a gene is epilepsy, an epilepsy syndrome, or an encephalopathy.

4. The method of claim 1, wherein the neurological disorder, the disorder associated with excessive neuronal excitability, or the disorder associated with a gain-of-function mutation of a gene is a genetic or pediatric epilepsy or a genetic or pediatric epilepsy syndrome.

5. The method of claim 1, wherein the neurological disorder, the disorder associated with excessive neuronal excitability, or the disorder associated with a gain-of-function mutation of a gene is a cardiac dysfunction.

6. The method of claim 1, wherein the neurological disorder, the disorder associated with excessive neuronal excitability, or the disorder associated with a gain-of-function mutation of a gene is chosen from epilepsy, encephalopathies, seizures, leukodystrophy, leukoencephalopathy, intellectual disability, Multifocal Epilepsy, drug-resistant epilepsy, Temporal lobe epilepsy, or cerebellar ataxia.

7. The method of claim 1, wherein the neurological disorder, the disorder associated with excessive neuronal excitability, or the disorder associated with a gain-of-function mutation of a gene is chosen from cardiac arrhythmia, Brugada syndrome, and myocardial infarction.

8. The method of claim 1, wherein the neurological disorder, the disorder associated with excessive neuronal excitability, or the disorder associated with a gain-of-function mutation of a gene is selected from pain and related conditions.

9. The method of claim 1, wherein the neurological disorder, the disorder associated with excessive neuronal excitability, or the disorder associated with a gain-of-function mutation of a gene is a muscle disorder.

10. The method of claim 1, wherein the neurological disorder, the disorder associated with excessive neuronal excitability, or the disorder associated with a gain-of-function mutation of a gene is chosen from itch and pruritis, ataxia, or cerebellar ataxias.

11. The method of claim 1, wherein the neurological disorder, the disorder associated with excessive neuronal excitability, or the disorder associated with a gain-of-function mutation of a gene is a psychiatric disorder.

12. The method of claim 1, wherein the neurological disorder, the disorder associated with excessive neuronal excitability, or the disorder associated with a gain-of-function mutation of a gene is chosen from a learning disorder, Fragile X, neuronal plasticity, or an autism spectrum disorder.

13. The method of claim 1, wherein the neurological disorder, the disorder associated with excessive neuronal excitability, or the disorder associated with a gain-of-function mutation of a gene is chosen from epileptic encephalopathy with SCN1A, SCN2A, and/or SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, sudden unexpected death in epilepsy (SUDEP), or KCNT1 epileptic encephalopathy.

14. The method of claim 1, wherein the subject is a human and the neurological disorder, the disorder associated with excessive neuronal excitability, or the disorder associated with a gain-of-function mutation of a gene is a R474H mutation in KCNT1.

15. The method of claim 14, wherein the R474H mutation in KCNT1 is a heterozygous mutation.

16. (canceled)

17. The method of claim 1, further comprising administering the compound of Formula (I-A) or a pharmaceutically acceptable salt thereof to the subject following birth.
